(19) United States
(12) Patent Application Publication
(10) Pub. No.: US 2014/0099287 A1
(43) Pub. Date: Apr. 10, 2014

(54) PLASMA PROTEIN CONCENTRATE FOR CELL DELIVERY IN REGENERATIVE APPLICATIONS

(71) Applicant: SpineSmith Partners, L.P., Austin, TX (US)

(72) Inventors: Matthew Murphy, Austin, TX (US); Theodore Sand, Austin, TX (US)

(73) Assignee: SpineSmith Partners, L.P., Austin, TX (US)

(21) Appl. No.: 14/046,995

(22) Filed: Oct. 6, 2013

Related U.S. Application Data
(60) Provisional application No. 61/710,704, filed on Oct. 6, 2012.

(51) Int. Cl. A61K 35/16

(52) U.S. Cl. CPC A61K 35/16
USPC 424/93.7; 435/29

(57) ABSTRACT

The invention is directed to concentrating autologously-derived plasma, using the concentrated plasma fluid to dilute the patient's cells and applying the combination of concentrated fluid with cells at a site of pathology or mixing the combination of concentrated fluid with cells with a particulate material like a bone void filler prior to placing the mixture at a site of pathology.

### CROSS-REFERENCES TO RELATED APPLICATIONS

This application claims the benefit under 35 U.S.C. §119(e) of U.S. Provisional Patent Application No. 61/710,704 filed Oct. 6, 2012, which is incorporated herein by reference in its entirety as if fully set forth herein.

### FIELD OF THE INVENTION

The invention involves concentrating autologously-derived plasma, using the concentrated plasma fluid to dilute the patient's cells and applying the combination of concentrated fluid with cells at a site of pathology.

### BACKGROUND OF THE INVENTION

Plasma protein concentrate (PPC) was investigated as a potential improvement for cell delivery in regenerative therapies. Blood plasma contains useful proteins for cell adhesion/retention including fibrinogen, fibronectin, and vitronectin. It is hypothesized that enriching these proteins' concentrations will enhance cell retention on biological substrates or at tissues injected with autologous cells. A second potential benefit of PPC is improved clotting. It has been clinically observed that bone marrow aspirate and bone marrow concentrate from a percentage of patients does not sufficiently clot when combined with a coagulation agent. PPC may increase fibrinogen and prothrombin concentrations to or above normal levels in deficient patients. For normal patients, increased fibrinogen and prothrombin is believed to result in more robust clots to minimize lost cells and fluid. PPC also contains growth factors, including platelet-derived growth factor (PDGF) and vascular endothelial growth factor (VEGF), which nourish cell functions at a higher concentration that normal plasma.

Plasma derived from blood or bone marrow contains many proteins with specific functions. Table 1 describes the most prevalent proteins found in plasma. Fibrinogen, fibronectin, vitronectin, and prothrombin are associated with natural wound repair mechanisms. Plasma also contains buffering and immune system proteins to maintain homeostasis of the circulating blood. Many growth factors (PDGF, VEGF, TGF-beta, and FGF) are responsible for cell recruitment and proliferation.

TABLE 1

| | Normal | | | |
|---|---|---|---|---|
| | Plasma | | | |
| | Concen- | | | Molecular |
| | tration | | Regenerative | Weight |
| Protein | (mg/mL) | Function | Application | (kDa) |
| Total | 65-80 | | | |
| Fibrinogen | 2.0-4.5 | Clotting | Faster clotting | 340 |
| | | | time, stronger | |
| | | | “bone logs” | |
| Fibronectin | 0.3 | Cell | Enhanced cell | 440 |
| | | migration/ | retention; | |
| | | adhesion, | Faster healing | |
| | | Wound closure | time | |
| Vitronectin | 0.2 | Cell adhesion | Enhanced cell | 75 |
| | | | retention | |
| Prothrombin | 0.05-0.1 | Clotting | Clotting rate | 72 |
| | | | control | |
| Albumin | 35-50 | Hormone | Buffer acidity | 67 |
| | | transport, | from carrier | |
| | | pH Buffer | breakdown | |
| Immuno- | 10-15 | Immune | | 150 (IgG)- |
| globulins | | recognition | | 900 (IgM) |
| VEGF | 0.0008 | Angiogenesis, | Faster blood | 40 |
| | | Endothelial | vessel growth | |
| | | cell migration | into graft site | |
| PDGF | 0.003 | Cell growth, | Stem cell | 31 |
| | | angiogenesis | replication, | |
| | | | faster blood | |
| | | | vessel growth | |
| TGF-β1 | 0.01 | Cell growth/ | Stem cell | 25 |
| | | differentia- | replication, | |
| | | tion | faster tissue | |
| | | | regeneration | |
| FGF | 0.0001 | Cell growth, | Stem cell | 20 |
| | | angiogenesis, | replication, | |
| | | Wound healing | faster blood | |
| | | | vessel growth | |
| SDF-1 | 0.0009 | Cell | Endogenous | 8 |
| | | recruitment/ | cell | |
| | | migration | recruitment | |
| RANTES | 0.002 | Cell recruit- | Endogenous | 8 |
| | | ment to | cell | |
| | | inflammation | recruitment | |
| | | site | | |

Clotting is a natural mechanism for wound closure and repair. Briefly, biomolecules signaling tissue damage are released by cells and platelets after injury. These molecules react with calcium to transform several clotting factors, culminating in the conversion of prothrombin to thrombin. Active thrombin cleaves portions of fibrinogen to form fibrin molecules, which are polymerized to form a fibrin clot. After clotting or coagulation, the wound undergoes stages of inflammation (restricted blood flow, recruitment of macrophages to remove foreign bodies and debris), proliferation (generation of new blood vessels, proliferation of cells, creation of new tissue, and contraction of the wound), and remodeling (cells convert fibrous tissue to a more mature and functional tissue).

### SUMMARY OF THE INVENTION

There is a problem with the delivery of regenerative cells and the environment in which they are applied, injected, sprayed or otherwise presented to a patient. Without taking special precautions, autologous regenerative cells might not remain at the site of application or treatment, thereby reducing their therapeutic potential. Standard approaches for retaining regenerative cells include allowing a cell preparation to soak into a bone void filler, usually in the form of granules (also can be used with block-shaped bone void fillers), but bone void fillers are not appropriate for all indications, especially those involving soft tissue pathologies. Another approach is to inject the cell preparation directly into a tissue pathology, e.g., into the capsular space of a joint like the knee, but there is no way to ensure that the cells will be retained on the articular surfaces. The invention addresses the need to improve delivery of cells for all kinds of pathologies, including bony and soft tissue pathologies. The invention involves concentrating autologously-derived plasma (from whole blood, bone marrow, etc.), using the concentrated plasma fluid to dilute the patient's cells and injecting or otherwise applying the combination of concentrated fluid with cells at a site of pathology. Alternatively, the concentrated fluid can be applied prior to the treatment with the patient's own cells, which will serve to coat the affected surfaces with proteins from the concentrated fluid, thereby improving the adherence of the cell preparation to the coated surfaces. There is a specific need to improve the retention by bone void fillers of autologous cells in order to improve the transfer of the cells into spinal fusion treatment sites in a patient. The invention specifically enhances cell retention of bone void fillers due to the formation of a clot within the particles of the bone void filler when placed into contact with the cell preparation and thrombin/CaCl₂. Improved clotting also will play a role in other pathologies, including the treatment of burns and topical wounds, in general, due to the formation of a clot containing cells and concentrated proteins and growth factors that coats the wound or burn site.

PPC may be used to “coat” biological substrates or carriers prior to the addition of cells in order to increase cell adhesion to those materials. It may also be used to “pre-coat” a tissue to be injected with cells for the same reason. In many instances, PPC may be co-delivered with cells. The PPC or PPC+cells also may be combined with a coagulation agent, such as thrombin or calcium chloride, to form a clot in situ or at the site of deposition. Delivery could be achieved through a syringe for applications such as disc or joint injections. Delivery could be achieved by spraying the solutions onto a surface for applications such as the treatment of skin burns. Increased cell retention and function is beneficial with most cell therapies including skeletal fractures, spinal fusion, intervertebral disc injections, joint injections, plantar fasciitis, torn cartilage, ligaments or tendons, wounds, burns, or ulcers of the skin, surgical closure of soft tissues, or internal organs.

### BRIEF DESCRIPTION OF THE DRAWINGS

FIG. 1 shows the fibrinogen deposition on tricalcium phosphate granules as measured by ELISA assay; and

FIGS. 2A and 2B show bone logs prepared with PPP that maintain their shape; and

FIG. 3 shows the cell retention by granular bone void fillers under several conditions.

### DETAILED DESCRIPTION OF EXEMPLARY EMBODIMENTS

In the broadest sense, the invention relates to the use of concentrated plasma (plasma protein concentrate, or PPC) as a diluent for suspending cells, and/or to coat tissue surfaces prior to cell application. More specifically, the invention relates to the concentration of autologous plasma (platelet-rich plasma or platelet-poor plasma) derived from peripheral blood or bone marrow aspirate and, subsequently, combining the concentrated autologous fluid preparation with autologous regenerative cells. PPC may be prepared from a patient's plasma by several methods including filtration, ultracentrifugation, cold precipitation, or lyophilization. The autologous regenerative cells may be derived from bone, bone marrow, adipose, dermis, or any combination thereof. Regenerative cells include mesenchymal stem cells, hematopoietic stem cells, stromal cells, pericytes, and endothelial progenitor cells, among others. PPC is enriched in plasma proteins including, but not limited to, fibrinogen, fibronectin, and vitronectin; and growth factors including, but not limited to, platelet-derived growth factor (PDGF) and vascular endothelial growth factor (VEGF). Use of an enriched autologous plasma-derived fluid may promote greater cell retention or adhesion, cell proliferation, cell migration for co-delivered cells, as well as chemotaxis and angiogenesis by local endogenous tissues, among other beneficial effects. Once the cells are combined with PPC, they may be delivered to a degenerated, injured, or diseased tissue for the purpose of tissue regeneration and/or modulation of inflammatory factors. Examples of these tissues include, among others, pathologies in bone voids, bone fractures, spinal fusions, intervertebral discs (restoration of disc height and rehydration), cartilage, ligaments, tendons, dermis, epidermis, skeletal muscle, cardiac muscle, lungs, liver, pancreas, kidneys, bladder. The invention may also be used to treat arthritis in cases where the tissue is not necessarily degenerated, injured, or diseased. The PPC may be applied to the pathologic sites in conjunction with and/or prior to treatment with autologous, regenerative cells. PPC may be applied in several ways, including injection through a needle, spraying onto a surface, or soaking onto a wound dressing or biomaterial. The PPC may be combined with a coagulant to form a fibrin matrix for increased cell retention. Examples of coagulants are thrombin (autologous, recombinant human, bovine, or porcine; concentration range 500-5000 units/mL, preferably 1000 units/mL) and a divalent cationic salt such as calcium chloride or calcium carbonate (concentration range 10-80 mM, preferably 20 mM).

PPC is prepared in several methods from platelet-poor plasma (PPP) derived from bone marrow aspirate. These methods included filtration through porous hollow microfibers (with and without a priming step) and ultrafiltration with centrifugation. The fibrinogen concentration was measured before concentration (PPP) and after concentration (PPC) in a hollow microfiber device.

The analysis also revealed substantial variability in plasma fibrinogen concentration from patient to patient. 20 unique plasma samples were analyzed from peripheral blood and bone marrow aspirate. Fibrinogen concentration ranged from 0.9 to 6.0 mg/mL with a standard deviation of 1.6. This wide variation could be responsible for insufficient clotting occasionally observed in the clinical setting. In the PPC, fibrinogen concentrations ranged from 8.0 to 13.4 mg/mL, indicating that even the most fibrinogen-deficient PPP sample had been enriched above the highest unconcentrated level.

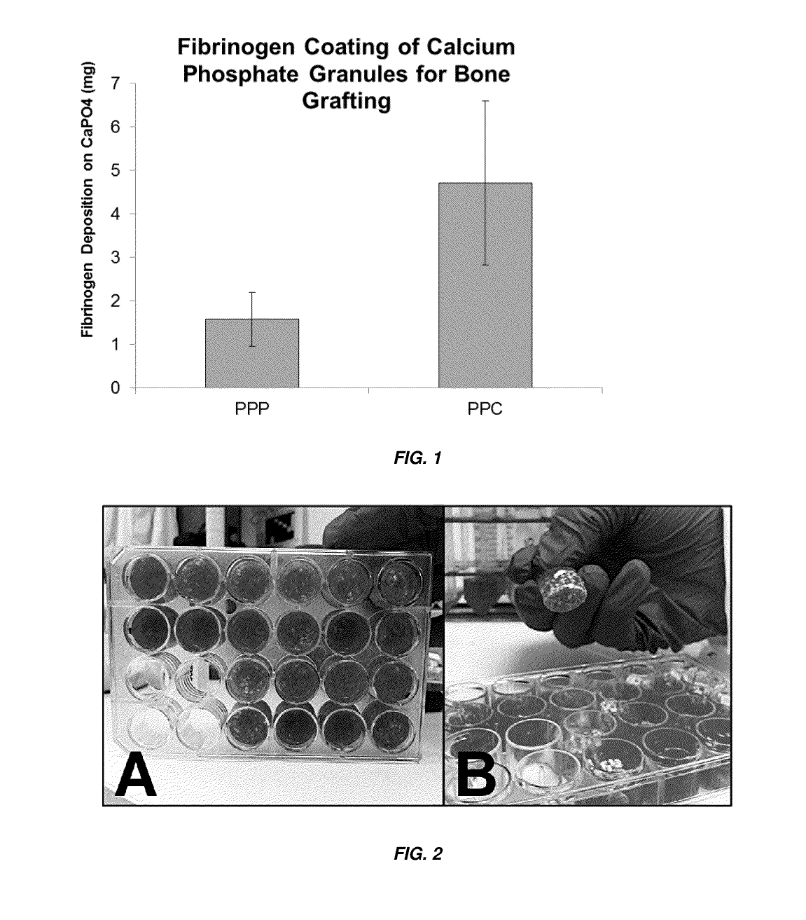

Next, the deposition of fibrinogen was examined as a model plasma protein, onto the surface of a tricalcium phosphate substrate (CymbiCyte, Celling Biosciences, Austin, Tex.). Briefly, 1 mL of substrate was coated with 1 mL of PPP or PPC for 10 minutes at 4° C. After incubation, the PPP or PPC was removed and the granules were washed with 1 mL saline to remove unbound proteins from the substrate. Fibrinogen deposition was measured for three PPP and PPC samples and the results are provided in FIG. 1. PPC deposited approximately three times as much fibrinogen onto the substrate than PPP. This increased protein deposition is hypothesized to promote higher cell adhesion and retention onto coated substrates and tissues.

Next, PPP or PPC was mixed with CymbiCyte in vitro and combined with coagulation agents (thrombin (bovine, 1000 units/mL) and calcium chloride (20 mM)) in situ in plastic molds. After 60 seconds, the formed “bone logs” were removed from the mold to evaluate sturdiness, handling properties, and unretained fluid. A representation of the bone logs are illustrated in FIGS. 2A and 2B.

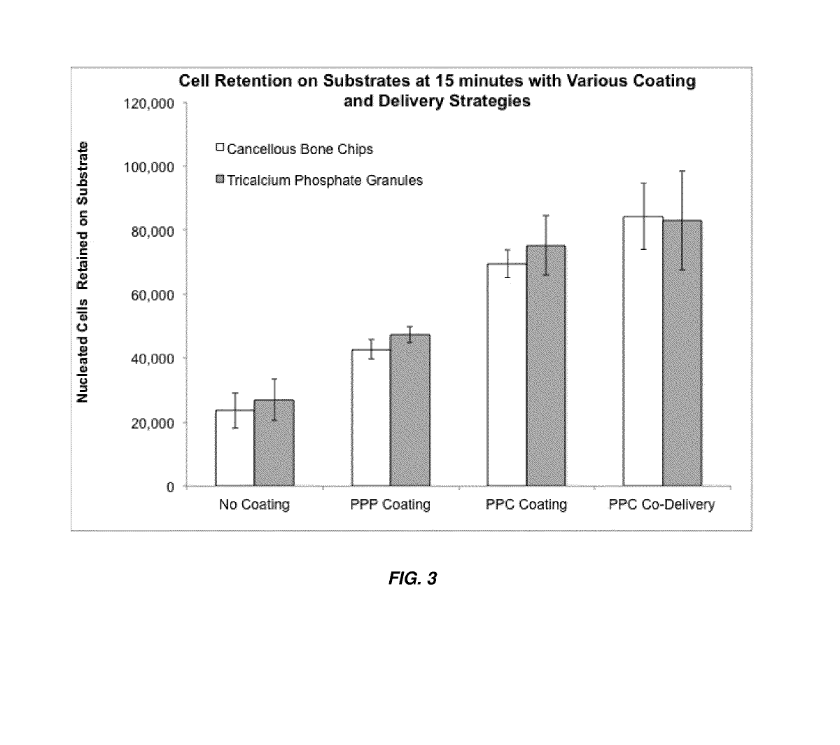

The interaction of PPC, bone void fillers (CymbiCyte and cancellous bone chips) and cultured cells was assessed. Cells were combined with the bone void fillers in the absence of PPC, after a PPP coating period, after a PPC coating period and finally with the cells mixed with the PPC before being combined with the bone void fillers. Retention of cells by the bone void fillers was highest for the PPC+Cell premixture, followed by PPC coating, PPP coating and with no coating having the lowest retention level. The cell retention values for the conditions are shown in FIG. 3.

PPC-Cell co-injection was demonstrated using PPC derived from peripheral blood plasma. Briefly, 2 mL of PPP or PPC was loaded into a 10 mL syringe. In a separate 1 mL syringe, 0.2 mL thrombin and calcium chloride mixture was drawn. The syringes were joined by a Y-connector and expelled through needles for joint (approximately 4 cm) or disc (approximately 10 cm) injections. Within approximately 30 seconds after injection, all PPC samples had formed a stable gel, while some PPP gels took 60 to 90 seconds to stabilize. Final gels of PPC were more firm, opaque, and lost less fluid than PPP gels. It is hypothesized that this will result in greater cell retention at the injection site using PPC compared to unconcentrated plasma, blood, or bone marrow.

### WORKING EXAMPLES

The below examples show the use of PPC with regenerative cells.

### Example 1

### Protein and Growth Factor Enrichment

Plasma proteins and growth factors may be enriched for increased dose when co-injected with cells. PPC was prepared by concentrated 30 mL PPP to 5 mL using hollow fiber tangential flow filters of two pore sizes. Protein and growth factor concentrations were measured by enzyme-linked immunosorbent assay (ELISA). The percentage of enrichment of beneficial plasma proteins above baseline (PPP) values using the two filters is listed in Table 2. Due to the smaller pore size of the 30 kDa unit, more proteins and growth factors were retained in the PPC compared to that prepared using the 60 kDa filter. Platelet-derived growth factor-AB/BB (PDGF-AB/BB), Transforming growth factor-beta 1 (TGF-b1), and Basic fibroblast growth factor (FGF-2) have been widely demonstrated in the literature to promote cell proliferation, migration, and differentiation that may be beneficial to the therapeutic effect when PPC is co-injected with regenerative cells.

Table 2 shows the percent increase in protein concentration of PPC prepared by 30 kDa and 60 kDa hollow fiber tangential flow filters compared to original PPP.

TABLE 2

| | 30 kDa Pore Size | 65 kDa Pore Size |
|---|---|---|
| Fibrinogen | 254% | 172% |
| PDGF-AB/BB | 447% | 180% |
| TGF-b1 | 420% | 260% |
| FGF-2 | 130% | 128% |

### Example 2

### Fibrinogen Coating of Biomaterial Substrates and Cell Retention

Coating biological substrates with adhesion proteins (fibrinogen, fibronectin, vitronectin) from plasma has advantages for cell adhesion/retention, providing molecular targets for cell binding. Five unique PPP and corresponding PPC samples (1 mL) each were used to coat 1 gram of a 60:40 hydroxyapatite-tricalcium phosphate (HA-TCP) granular substrate. Fibrinogen deposition onto the tricalcium phosphate granules was measured by ELISA assay (results shown in FIG. 1). On average, at least 3 times the mass of fibrinogen was deposited onto the biomaterial from an equal volume of PPC compared to PPP from the same donor blood sample.

The benefit of pre-coating or co-delivering cells with PPC was demonstrated by observing cell retention on common orthopedic bone graft substrates in vitro. Cancellous bone chips or tricalcium phosphate granules (0.5 mL each) were untreated or pre-coated with 0.5 mL PPP or PPC for 15 minutes. After coating, the PPP or PPC was drained from the substrates and a solution of 700,000 bone marrow mesenchymal cells in 1 mL of buffered medium was applied. A fourth experimental group consisted of co-delivering the cell solution with an equal volume to PPC to uncoated bone chips or tricalcium phosphate granules. Each variable was tested in triplicate (n=3). After a 15 minute adhesion period, the cell solution was removed from each sample and the cells were counted. The average number retained cells and standard error are reported in Table 3. There is a statistically significant increase in retained cells on both types of substrates by using PPC as a coating or co-delivery agent compared to uncoated or PPP-coated materials.

TABLE 3

| | | | Tricalcium Phosphate | |
|---|---|---|---|---|
| | Cancellous Bone Chips | | Granules | |
| | Number of Cells | Fold | Number of Cells | Fold |
| Condition | Retained | Increase | Retained | Increase |
| No Coating | 2.37 | 1.0 | 2.70 | 1.0 |
| | (±0.56) × 10⁴ | | (±0.65) × 10⁴ | |
| PPP | 4.27 | 1.80 | 4.73 | 1.75 |
| Pre-coating | (±0.30) × 10⁴ | | (±0.24) × 10⁴ | |
| PPC | 6.95 | 2.94 | 7.52 | 2.78 |
| Pre-coating | (±0.44) × 10⁴ | | (±0.93) × 10⁴ | |
| PPC | 8.43 | 3.56 | 8.30 | 3.07 |
| Co-Delivery | (±0.10) × 10⁴ | | (±0.16) × 10⁴ | |

### Example 3

### Clot Stability

BMC, PRP, or PPP activated with 10% calcium chloride and/or thrombin forms a clot that is usually unstable and without mechanical strength sufficient to resist deformation under stress. Sample of PPP and PPC from matching donors were activated with 10% calcium chloride and thrombin by mixing through a dual syringe and “Y” connector to form 1 cc spherical clots. Clots formed from PRP were partially transparent and released approximately 20% of their fluid volume under mild compression. Conversely, clots formed from PPC were opaque (indicating a denser network of proteins) and lost no more than 5% of their fluid volume under mild compression. The loss of fluid is analogous to a loss of regenerative cells in vivo after injection.

### Example 4

### Injection of Autologous PPC and Regenerative Cells for Osteoarthritis Treatment And Articular Cartilage and Meniscus Repair

To increase cell retention at the site of injection by means of increased protein content, autologous regenerative cells were prepared at the point-of-care and mixed with autologous PPC for percutaneous injection into arthritic or damaged tissues. In one instance, 5 mL bone marrow concentrate (BMC) prepared from 60 mL bone marrow aspirate (BMA) and 5 mL PPC prepared from 30 mL PPP were co-injected into arthritic knee and hip joints for the cumulative and synergistic benefits of BMC, platelets, and plasma proteins and growth factors. In a different application, articular cartilage damage and osteoarthritis of the knee was treated with a combination of mononuclear cells harvested from the patient's bone marrow (4 mL) and stromal vascular fraction of adipose (4 mL) were injected bilaterally into the knee capsule after injection of PPC (6 mL) to wash the joint and coat the cartilage surfaces with proteins. For treatment of partially torn meniscus, 5 mL autologous BMC was mixed with 5 mL PPC and activated with 1 mL 10% calcium chloride and thrombin prior to arthroscopic injection into the damaged site. In each of these cases, surgeons indicated improved patient outcomes compared to the conventional standard of care for the respective orthopedic applications.

### Example 5

### PPC as a Carrier for Cellular Injection Therapy for Degenerative Disc Disease

Degenerative disc disease describes pain associated with damaged, dehydrated/desiccated, herniated, or depressed intervertebral discs. Current treatment options include rest, steroid or anti-inflammatory injections, discectomy, and/or spinal fusion surgery. In the case of tears or herniation, fluid from the nucleus pulposus may escape the disc and contact a nerve or the spinal cord, resulting in severe back pain. In a pilot study, 3 mL autologous BMC and PPC was prepared from 60 mL BMA and injected into degenerated lumbar intervertebral discs of patients were fusion surgery candidates. Intervertebral disc injection with BMC resulted in an average reduction of pain scores of 57% (ODI) and 65% (VAS) at 3 months post-therapy, 58% (ODI) and 72% (VAS) at 6 months, and 62% (ODI) and 63% (VAS) at 12 months compared to pre-injection pain scores. PPC provided growth factors for cell proliferation and bioactivity and aided in the repair of annular tears.

### Example 6

### Spinal Fusion Graft Preparation

In spinal fusion surgeries, graft materials are implanted to regenerate bone to fuse adjacent vertebral bodies. For many biomaterial substrates, their physical properties are not sufficient to form moldable grafts for interbody or posterolateral fusion. Many ortho-biologic graft materials do not possess favorable surface characteristics for cell adhesion/retention, growth, and differentiation. Surgeons often form grafts as “bone logs” using particles or granules glued together by soaking the materials in and clotting the patient's PRP or bone marrow. Because approximately 1 in 8 patients are deficient in clotting proteins, it is not always possible to form these types of grafts. Two popular graft materials (cancellous bone chips and tricalcium phosphate granules) were formed into bone logs using PRP and PPC in an in vitro setting. In the PRP-soaked bone logs, 7 of 12 grafts maintained their shape after two minutes. Bone logs prepared with PPP maintained their shape after two minutes in 11 of 12 grafts (FIG. 2). In posterolateral spinal fusion surgery, grafts were prepared with tricalcium phosphate and hydroxyapatite granules (10 mL) and PPC (5 mL) derived from the patients BMA. After activation with 1 mL 10% calcium chloride and thrombin, sturdy bone logs were formed and implanted for successful spinal fusion.

FIG. 2A shows bone grafts that are formed with cancellous bone chips or tricalcium phosphate granules and PPC. FIG. 2B shows that PPC-based bone grafts hold together and retain their shape. PPC bone grafts are more robust than grafts formed with PRP and retain their shape under stress with greater frequency than PRP-based grafts.

### Example 7

### Rotator Cuff Surgery

Rotator cuff injuries include tears and detachments of muscles and ligaments in the shoulder joint. Many surgeons wish to augment the standard clinical treatment methods with regenerative cells but lack an appropriate carrier to delivery the cells. In one instance, a partially torn rotator cuff may be treated by percutaneous injection of cells mixed with PPC and activated by a clotting agent such as calcium chloride and/or thrombin. Upon injection, the PPC acts as a biologic glue to bridge the tear and retain the cells. In another instance, rotator cuff repair surgery may utilize regenerative cells by gluing the cells at the tendon insertion site after surgically fixing the tendon to the bone by anchor, screw, suture, or another implant.

What is claimed is:

1. A method of preparing an enriched plasma-derived fluid, the method comprising:
preparing plasma protein concentrate;
preparing regenerative cells; and
combining the plasma protein concentrate with the regenerative cells to form an enriched plasma-derived fluid.

2. The method of claim 1, wherein the plasma protein concentrate is prepared by filtration, ultracentrifugation, cold precipitation or lyophilization.

3. The method of claim 1, wherein the regenerative cells are derived from bone, bone marrow, adipose, dermis or any combination thereof.

4. The method of claim 1, wherein the plasma protein concentrate is derived from peripheral blood or bone marrow aspirate.

5. A method for improving cell adhesion and retention on a substrate, the method comprising the steps of:
coating plasma protein concentrate on a surface of the substrate;
applying cells or protein on the coated substrate; and
assessing the adhesion and retention of the cells or protein on the coated substrate.

6. The method of claim 5, wherein the plasma protein concentrate is prepared by filtration, ultracentrifugation, cold precipitation or lyophilization.

7. The method of claim 5, wherein the regenerative cells are derived from bone, bone marrow, adipose, dermis or any combination thereof.

8. The method of claim 5, wherein the plasma protein concentrate is derived from peripheral blood or bone marrow aspirate.

9. A method for improving cell adhesion and retention in a treatment site, the method comprising;
preparing an enriched plasma-derived fluid;
introducing the enriched plasma-derived fluid to a treatment site; and
applying regenerative cells or proteins to the treatment site.

10. The method of claim 9, wherein the plasma protein concentrate is prepared by filtration, ultracentrifugation, cold precipitation or lyophilization.

11. The method of claim 9, wherein the regenerative cells are derived from bone, bone marrow, adipose, dermis or any combination thereof.

12. The method of claim 9, wherein the plasma protein concentrate is derived from peripheral blood or bone marrow aspirate.

13. A method for improving cell adhesion and retention in a treatment site, the method comprising;
preparing an enriched plasma-derived fluid;
preparing regenerative cells;
mixing the enriched plasma-derived fluid with the regenerative cells; and
introducing the mixture of the enriched plasma-derived fluid and regenerative cells to a treatment site.

14. The method of claim 13, wherein the plasma protein concentrate is prepared by filtration, ultracentrifugation, cold precipitation or lyophilization.

15. The method of claim 13, wherein the regenerative cells are derived from bone, bone marrow, adipose, dermis or any combination thereof.

16. The method of claim 13, wherein the plasma protein concentrate is derived from peripheral blood or bone marrow aspirate.

17. A method for improving cell adhesion and retention in a treatment site, the method comprising;
preparing a particulate material;
preparing regenerative cells;
mixing the particulate material with the regenerative cells; and
introducing the mixture of the enriched plasma-derived fluid and regenerative cells to a treatment site.

18. The method of claim 17 wherein the particulate material is bone void filler.
